(19) United States
(12) Patent Application Publication
(10) Pub. No.: US 2022/0346333 A1
(43) Pub. Date: Nov. 3, 2022

(54) STORAGE AND CURING RACKING SYSTEM FOR PLANT PRODUCTS

(71) Applicant: SOUTHWESTERN LEGISLATIVE SAFETY CONSULTING INC., Windsor (CA)

(72) Inventor: Darren Varga, LaSalle (CA)

(21) Appl. No.: 17/734,682

(22) Filed: May 2, 2022

Related U.S. Application Data
(60) Provisional application No. 63/182,197, filed on Apr. 30, 2021.

(51) Int. Cl. A01G 31/06; A01G 7/04; A01G 24/46; A01H 6/28; G01N 27/12; F24F 11/30

(52) U.S. Cl. CPC A01G 31/06; A01G 7/045; A01G 24/46; A01H 6/28; G01N 27/121; F24F 11/30; F24F 2110/20

(57) ABSTRACT

A storage racking system for plant products can be capable of automated rotation and curing of dried cannabis and hemp flowers in sealed containers, which can maintain relative humidity along with a UV light and oxygen deficient environment to reduce degradation. The storage containers can be positioned horizontally to maintain a connection to the inert gas connection and allow for daily rotation in either direction. Each storage container can have a lid and a humidity pack. The lid can have a pressure relief valve that can exchange the gaseous atmosphere from the storage container with an inert gas through a check valve connection. The humidity pack can maintain the relative humidity in the storage container at about 58% to 62% during the curing and storage stages.

### CROSS-REFERENCE TO RELATED APPLICATIONS

This application claims the benefit of U.S. Provisional Application No. 63/182,197, filed on Apr. 30, 2021. The entire disclosure of the above application is incorporated herein by reference.

### FIELD

The present disclosure relates to storing and curing various plant products and, more particularly, to an automated system for storage and curing cannabis.

### INTRODUCTION

This section provides background information related to the present disclosure which is not necessarily prior art.

Plant material, such as cannabis and hemp flowers, is perishable. As soon as a cannabis plant is harvested, for example, it begins to degrade. Not only is the plant itself no longer alive and receiving nutrients from the root ball it was once attached to, but the cannabinoid biosynthetic pathways are disrupted as well. In this process, cannabinoids and terpenes synthesize into other compounds, subsequently altering their properties, which can include their psychoactive properties. Once a cannabis plant has been harvested, dried, and cured, its optimal freshness zone becomes a finite window that can be extended only by carefully controlling various elements that include not only temperature and relative humidity, but also ultraviolet (UV) light exposure and ambient oxygen levels.

Storage and curing of cannabis and hemp are integral parts of a fast-growing cannabis and hemp industry. Storage and curing systems are a costly part of cannabis and hemp processing, and if done incorrectly, can reduce the quality and salability of the product. This process normally is completed by manually opening a storage container to relieve off-gassing during the curing process of dried cannabis and hemp flowers, and further manually rotating dried cannabis and hemp flowers mechanically with a device or with one's hand. This process is recognized in the industry as “burping” the product during the storage and curing stages.

The manual burping process requires constant care by staff to ensure that dried cannabis and hemp flowers do not degrade too quickly. Degradation of the product can be caused by not burping the storage container frequently enough, which can allow a buildup of gases released by the dried flower as it cures. This can lead to contamination of the dried flower.

Another cause of dried flower degradation includes over exposure to UV light and oxygen during the storage and curing stage. Exposure to UV light and oxygen can cause the highest rates of degradation in the shortest time frame. UV light rays can break down organic matter at a rapid rate, causing cannabinoid degradation and loss to occur. Likewise, oxygen can result in oxidation of cannabinoids and other compounds, altering the chemical profile of the material. Such degradation can only be slowed by limiting UV light and oxygen exposure to cured flowers.

Another issue with traditional curing systems relates to controlling humidity. High relative humidity in and of itself can affect cannabinoid degradation by introducing high levels of moisture back into the flowers. Harvested cannabis and hemp experience a rapid slowing of fluid transfer during the drying and curing phase. Optimally, dried flower should be stored with a relative humidity in the 58-63% range to avoid adverse degradation in either direction. With a reintroduction of high amounts of moisture, cannabis and hemp flowers not only risk mold growth, but can also bring along ammoniated flavors due to the restricted air circulation. On the other end, low humidity can negatively impact cannabis by drying leaves and foliage out, causing the plant material to become undesirably brittle and fragile.

To summarize, temperature, humidity, airflow, and light are four major factors that influence the degradation of dried cannabis and hemp flowers. All four variables represent a spectrum that harvested cannabis and hemp flowers rest on. Controlling these variables and maintaining their proper levels can significantly prolong a shelf life of cannabis and hemp flowers by limiting exposure to the processes that influence the degradation of not only the floral clusters themselves, but the cannabinoids and terpenoids contained within them. Although cannabinoid degradation is an inevitability, and preferable characteristics of cannabis and hemp flowers will eventually expire at some point, maintaining a highly controlled environment is the best way to combat the natural processes that can render products undesirable, leaving fresh flowers for longer than may have previously anticipated.

Accordingly, there is a continuing need for ways of storing and curing plant matter that desirably militate against cannabinoid degradation.

### SUMMARY

In concordance with the instant disclosure, systems and methods for storing and curing plant products, which can militate against cannabinoid degradation, have been surprisingly discovered.

The present technology relates to a racking system for a plant product that can include a container having a lid, a fluid inlet, a valve, and an agitator. The lid can be configured to fluidly seal the container. The fluid inlet can be configured to introduce a fluid into the container. The valve can be configured to release the fluid from the container. And the agitator can be configured to impart a motion unto the container.

In certain embodiments, a racking system for a plant product is provided that includes a plurality of containers, where each container can include a lid configured to fluidly seal the container. A fluid inlet can be disposed in each lid, where the fluid inlet can be configured to introduce a fluid into the container, and where the fluid inlet can include a check valve. An inert gas source can be provided that is configured to be fluidly coupled to each fluid inlet to provide the fluid. A valve can be disposed in each lid, where the valve can be configured to release the fluid from the container, and where the valve can include a pressure release valve. An agitator can be provided that is configured to impart a motion unto each container. A screen can be configured to be disposed within each container and prevent the plant product from contacting the lid when the lid fluidly seals the container. A humidifying element can be configured to be disposed within each container when the lid fluidly seals the container. A controller can be configured to control the agitator and the motion imparted unto each container. A rack can be provided that is configured to dispose the plurality of containers in one or more horizontal rows and/or vertical columns.

The present technology further provides various ways of storing and curing a plant product that can employ one or more racking systems as provided herein. In particular, the plant product can be placed into one or more containers, where the lid of the container(s) is used to fluidly seal the container(s). The fluid can be introduced into each container through the fluid inlet. The motion can be imparted unto the container using the agitator. And the fluid can be released from the container(s) through the valve.

It is therefore possible to store and cure plant material, such as dried cannabis and hemp products, using the present technology. The provided racking systems and methods can be automated and can be capable of automated daily rotation and curing of dried cannabis and hemp flowers in sealed containers, which can maintain a desired or predetermine relative humidity along with a UV and oxygen deficient environment. This can ensure longevity of the plant product by reducing degradation thereof. Interchangeable storage containers can be positioned in the storage rack system to maintain the dried cannabis and hemp flower at optimal conditions to slow cannabinoid degradation. Variable sized storage containers can be manufactured from opaque food grade plastic or metal. The variable storage containers can be positioned horizontally to maintain a connection to an inert gas connection and allow for daily rotation in different directions. Each storage container can have a lid configured with a threaded pressure relief valve that can relieve the gaseous environment from the storage container, which can include off gassing by the plant product as carried by the fluid (e.g., inert gas). Protruding through the container lid can be a quick disconnect fluid line that provides an inert gas to purge oxygen and off-gassing out of the pressure relief valve during the curing stage. The storage container lid can also include a humidity pack that can maintain a relative humidity in the storage container at about 58% to 62% during the curing and storage stages.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates a front plan view of a storage and curing racking system for a plant product;

FIG. 2 illustrates a top perspective view of the racking system;

FIG. 3 illustrates a left side elevational view of the racking system;

FIG. 4 illustrates a top perspective view of a storage container that can have a rotational motion applied thereto;

FIG. 5 illustrates right side elevational view of the storage container;

FIG. 6 illustrates a top perspective view of a drive wheel of an agitator configured to impart rotational motion unto the container;

FIG. 7 illustrates an enlarged partial perspective view of drive shafts coupled via miter gear drive connections to translate power from a power source to the agitator;

FIG. 8 illustrates another enlarged partial perspective view of drive shafts coupled via miter gear drive connections to translate power from a power source to a drive wheel of the agitator;

FIG. 9 illustrates an enlarged partial perspective view of a drive motor coupled to a vertical drive shaft via a chain;

FIG. 10 illustrates an exploded view of a container and related components of the racking system;

FIG. 11 illustrates a cross-sectional view of the storage container and related components of the racking system; and

FIG. 12 illustrates a flowchart of a method of storing and curing a plant product using one or more embodiments of the racking system.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology relates to ways of optimizing the storage and curing of plant products in order to maintain desirable characteristics, for example, by preventing breakdown or modification of the plant products that may result from UV and oxygen exposure. What is more, a desired relative humidity can be maintained to prevent over drying or under drying of the plant product and the plant product can be agitated to mix the plant product to provide uniform exposure to a controlled environment and promote uniform curing thereof. Agitation of the plant product can occur based upon a predetermined schedule relative to agitation events, timing thereof, and duration thereof, in order to optimize or tailor the curing of a particular plant product for desired product characteristics. It should be recognized that while cannabis and hemp plant products are used as examples herein, the present technology can be applied to various plant products other than cannabis and hemp, where such other plant products can include various agricultural and food products, including herbs, spices, teas, seeds, vegetables, fruits, and other products that may benefit from minimizing UV and oxygen, maintaining a desired humidity, and providing a means for off gassing of the product during a curing process. Particular reference is made to cannabis and hemp herein, as these plant products have shown to exhibit substantial and surprising benefits when subjected to the present technology.

In certain embodiments, a racking system for a plant product is provided that can have a container, a lid, a fluid inlet, a valve, and an agitator. The lid can be configured to fluidly seal the container. The fluid inlet can be configured to introduce a fluid into the container. The valve can be configured to release the fluid from the container. The agitator can be configured to impart a motion unto the container.

The container including the lid configured to fluidly seal the container can include the following various aspects. The container can include a material that blocks a portion of ultraviolet light from passing therethrough. For example, where the container is formed using a plastic material, one or more UV absorbers can be included in the plastic material. The container can also be configured to block substantially all UV light from passing into the container. Certain materials can be used in manufacture of the container, including various plastics, metals, glass, and composite materials. Particular materials include polyolefins, such as polypropylene and polyethylene, where such polymers can include UV absorbers. Other materials include aluminum and composite materials, such as polymers including metallized polymers; e.g., BoPET (biaxially-oriented polyethylene terephthalate) and metallized BoPET. The container can also be made of a material that is substantially resistant to gas transmission, including being substantially impermeable to oxygen from ambient air.

The container can be formed in various shapes and sizes dependent on the amount of plant material to be placed therein, as well as a desired amount of void space, which can be related to how the container and its contents are going to respond to motion from the agitator. Examples include where the container includes a generally rigid structure with a removable lid or where the lid is integral to the container and can be reversibly opened and closed. The lid can be completely removable, tethered to the container in some fashion, mechanically coupled to the container (e.g., a hinged connection), and can comprise a portion of the container or be integral to the container where the lid allows reversible access to an interior of the container. The container can also take a flexible form and can be configured as a flexible plastic bladder, pouch, or bag, where the lid can be configured as a closure for the container, whether an integral closure or a removable closure. It is possible that the container can be at least partially inflated upon introduction of the fluid via the fluid inlet. The container and lid can include cooperating mechanical sealing mechanisms, where for example, the container and lid (including where the lid is integral to the container) can include a zipper-like mechanism or a slider mechanism. Examples include reclosable mechanisms similar to those found on fluid tight resealable bags and containers marketed under the tradename ZIPLOC and related reversible fluid tight sealing mechanisms using various sliders and the like.

Certain embodiments include where the container is substantially cylindrical and where the container can be configured generally as a bucket. Particular examples include standard 5-gallon and 7-gallon food grade plastic buckets. In this way, the container can be rotated to mix or turn over contents therein. It can be desirable to agitate the container so that the plant product contents are exposed to a relatively uniform and controlled environment during the curing process. The container can further include various baffles, tumbling media, and/or agitation means that can facilitate the mixing and manipulation of contents by the motion imparted by the agitator. The lid used in conjunction with the container can be configured to engage the container to form a fluid tight seal. The lid and/or the container can include one or more O-rings or seals formed of various elastomeric materials in order to fluidly seal the lid relative to the container.

The fluid inlet configured to introduce the fluid into the container can include the following various aspects. It is possible to configure the fluid inlet to introduce the fluid into the container in various ways and with various functionalities. In certain embodiments, the fluid inlet can be disposed in the lid and the fluid inlet can include a check valve, which only allows the flow of fluid therethrough in one direction, and which is also commonly referred to as a non-return valve or a one-way valve. The check valve can prevent the fluid introduced therethrough from backflow toward the fluid source, thereby preventing any off gassing from the plant product or other volatiles or humidity from being introduced into the fluid source. The fluid inlet can be disposed relative to the container or lid in a way that is compatible with the motion imparted unto the container by the agitator. For example, the fluid inlet can be disposed in line with an axis of rotation of the container imparted by the agitator, thereby minimizing movement, twisting, or tangling of any fluid coupling (e.g., tubing) attached to the fluid inlet. To this end, the fluid inlet can include an infinitely rotatable coupling that can minimize or prevent movement or twisting of the fluid coupling to the fluid inlet. Certain embodiments include where the fluid inlet includes a check valve, an infinitely rotatable coupling, and is disposed in a center of the lid, thereby allowing a cylindrical container fluidly sealed thereby to rotate through any number of revolutions imparted by the agitator. A similar configuration, without the infinitely rotatable coupling, can still permit the container to rotate through multiple revolutions (e.g., 540 degrees) in either direction without unduly twisting or kinking any fluid coupling (e.g., tubing) attached to the fluid inlet.

Various types of inert gas sources can be fluidly coupled to the fluid inlet. Examples include inert gases and mixtures thereof having argon, nitrogen, and/or carbon dioxide. The inert gas should be substantially inert relative to the plant product to stored and cured by the system. That is, the inert gas should be chosen such that it will not react with the plant product. One skilled in the art can ascertain what inert gas or mixture of inert gas can minimize undesirable reaction(s) with a given plant product. For cannabis and hemp, for example, oxygen or air (e.g., about 21% oxygen) can result in undesirable reactions, including oxidation, of various compounds (e.g., cannabinoids) in the plant product. The inert gas can include and/or can be mixed with water vapor to provide a certain humidity level into the container. In certain embodiments, the inert gas can have essentially no water vapor; e.g., bone-dry nitrogen or carbon dioxide, where other embodiments the inert gas can include or be mixed with water vapor to provide certain humidity ranges, including from about 50% to about 70%, from about 55% to about 65%, and from about 58% to 62% humidity. The inert gas source can be fluidly coupled to the fluid inlet in various ways, using various valving, junctions, filters, conduits, and tubing. For example, the inert gas source can be fluidly coupled to the fluid inlet using self-retracting tubing or pigtail tubing that can minimize effects of any twists or kinks imparted thereto. Such tubing can be formed of various flexible polymers and elastomers.

The valve configured to release the fluid from the container can include the following various aspects. It is possible to configure the valve to operate as an outlet for the fluid (and any off gassing from the plant product) from the container in various ways and with various functionalities. In certain embodiments, the valve can include a pressure release valve, also referred to simply as a relief valve, which can control a pressure build up in the container and provide an outlet to the fluid upon attainment of a predetermined pressure value. The valve can be disposed in the lid of the container. It is further possible to provide a fluid coupling to the valve to collect and/or direct the fluid (and any off gassing from the plant product) from the container. In this way, for example, the fluid (and any off gassing from the plant product) released from the container can be vented, filtered, dehumidified, recycled, and/or directed to a desired location.

The agitator configured to impart the motion unto the container can include the following various aspects. The agitator can be configured to engage the container in various ways in order to move or mix the plant product contained therein. In this way, the plant product is not subjected to environmental gradients or different microclimates within the container, where the motion tumbles or mixes the plant product so that an entirety of the plant product more uniformly experiences the controlled environment provided in the container, promoting uniform curing of the plant product. In certain embodiments, agitator can be configured to impart a rotary motion unto the container. For example, the agitator can include a drive wheel configured to contact the container and impart the rotary motion unto the container. The drive wheel can be configured to receive a portion of a weight of the container, along with one or more other non-powered wheels, so that the container can rest upon the wheels. The non-powered wheel(s) can be configured to cooperate with the drive wheel to receive another portion of the weight of the container. The weight of the container can provide sufficient contact and engagement with the drive wheel. Certain embodiments include where the container is substantially cylindrical and placed into contact with the drive wheel with the cylindrical axis substantially horizontal; e.g., where the container is a bucket and is laid upon its side onto the drive wheel and associated non-powered wheels.

The agitator can be powered by a central power source, such as an electric motor, where the power source can be coupled to one or more agitators in various ways. For example, the power source can include an electric motor coupled to one or more drive shafts that can impart the motion unto the container; e.g., via the aforementioned drive wheel. Various drive shafts can be mounted using drive shaft bearings to a rack or scaffolding and can be coupled by various miter gear drive connections, which can allow directional changes in motion transmission. In this way, a single drive motor (e.g., electric motor) can engage a drive shaft (e.g., via a chain drive) and the drive shaft can transmit motion to other drives shafts (e.g., via miter gear drive connections). Such configurations can be used to power multiple agitators that are each configured to impart motion unto a respective container. Various numbers of agitators for horizontally and/or vertically arranged or racked containers can be powered in this manner. Such arrangements can allow substantially identical motion to be imparted unto each container, thereby allowing the plant product in each container to be subject to a relatively uniform experience.

Embodiments of the racking system can further include the following various aspects. A screen can be included that is configured to be disposed within the container and prevent the plant product from contacting the lid when the lid fluidly seals the container. In this way, the screen can prevent the plant product from interfering with the fluid inlet and the valve when each are disposed within the lid. The fluid can therefore be introduced and released from the container without obstruction by the plant product, especially where the screen keeps the plant product spaced from the lid during and following motion imparted unto the container by the agitator. A humidifying element can be included that is configured to be disposed within the container when the lid fluidly seals the container. The humidifying element can be an active humidifying element or a passive humidifying element. Examples of active humidifying elements include powered or electrical humidifying units, including various heated units and moisture exchange units. Examples of passive humidifying elements include various wicking materials that can be prewet, including various porous media that can hold a solution including water (e.g., 50/50 water and propylene glycol) allowing passive evaporation of water vapor therefrom. Other examples include salt and water packs, crystal gels, and silica gel beads that can be configured to provide a certain humidity for a certain volume based upon the container size. The humidifying element can be coupled to an interior face of the lid, where the humidifying element can be reversibly coupled allowing replacement and/or recharging or rewetting of the humidifying element, as necessary. In conjunction with the screen, when the humidifying element is coupled to the lid of the container, the screen can prevent the plant product from contacting and interfering with the humidifying element.

The racking system can further include a controller having the following various aspects. The controller can be configured to control the agitator and the motion imparted unto the container. The controller can be programmable and can include a memory and a processor. The memory can include any type suitable to the local application environment and can be implemented using any suitable volatile or nonvolatile data storage technology, such as a semiconductor-based memory device, a magnetic memory device and system, an optical memory device and system, fixed memory, and removable memory. For example, the memory can include one or more of random-access memory (RAM), read-only memory (ROM), static storage such as a magnetic or optical disk, a hard disk drive (HDD), or any other type of non-transitory machine or computer-readable media. Instructions stored in the memory can include program instructions or computer program code that, when executed by one or more processors, enable the controller to operate the racking system, including the power source and power transmission to one or more agitators, as described herein. In certain embodiments, the controller can be interfaced using a computer or can embody a computer, and therefore can provide a user interface for programming automatic and/or manual operation of the racking system. The controller can also be in communication with one or more remote devices (e.g., mobile phones, tablets, laptops, etc.), through wired and/or wireless communication, where the controller and/or associated computer(s) can report the operational status of the racking system as well as receive automated instructions, manually inputted instructions, or instruction modifications.

The racking system can further include where the container is one of a plurality of containers, with each container including a lid configured to fluidly seal the container. In this way, each respective container and lid can be configured with a respective fluid inlet and a respective valve, where a respective agitator can be configured to impart a motion unto the respective container. That is, the fluid inlet can be one of a plurality of fluid inlets, where each fluid inlet can be configured to introduce a fluid into a respective container. Likewise, the valve can be one of a plurality of valves, where each valve can be configured to release the fluid from a respective container. And the agitator can be one of a plurality of agitators, where each agitator can be configured to impart a motion unto a respective container. A rack or scaffolding can be configured to dispose the plurality of containers in an arrangement including one or more horizontal rows and/or one or more vertical columns.

The present technology further provides various methods of storing and curing a plant product that can employ one or more of the racking systems as described herein. Such methods can include placing the plant product in the container. The container can then be fluidly sealed with the lid. The fluid can be introduced into the container through the fluid inlet. The motion can be imparted unto the container using the agitator. And the fluid can be released from the container through the valve.

As described, storage and curing of dried cannabis and hemp flowers using automated rotation, atmospheric purging, and relative humidity adjustment are provided herein. In certain examples, the present technology can be used in combination with a software system. The controller and any associated software can run the storage and curing system to rotate and purge the container internal atmosphere daily. The controller and any associated software can therefore provide an automated system that can be modified and adapted to the plant product and/or to the installation requirements of a particular storage room, area, or facility.

### EXAMPLES

Example embodiments of the present technology are provided with reference to the several figures enclosed herewith.

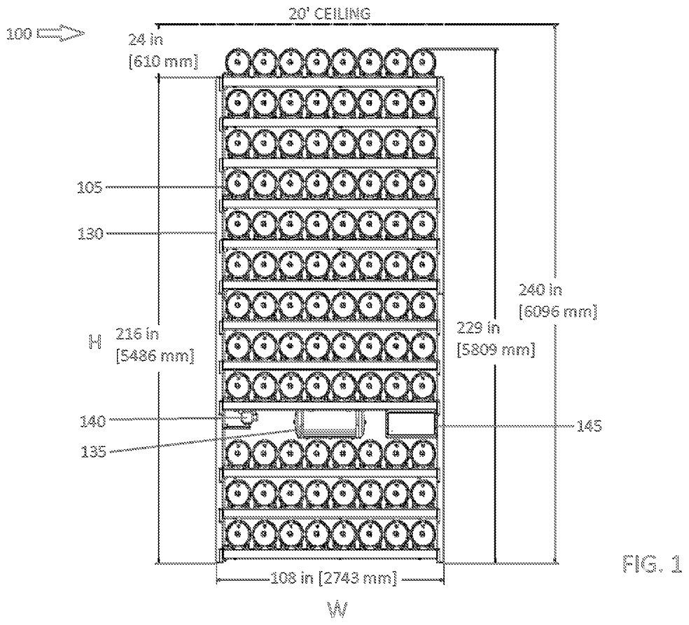

With reference to FIGS. 1-11, shown is a storage and curing racking system 100 for a plant product and related components thereof, as constructed in accordance with the present technology. The racking system 100 includes multiple containers 105, where each container 105 has an associated lid 110, a fluid inlet 115, a valve 120, and an agitator 125. Each lid 110 can be configured to fluidly seal the container 105. Each fluid inlet 115 can be configured to introduce a fluid into the container 105. Each valve can be configured to release the fluid from the container 105. Each agitator 125 can be configured to impart a motion unto the container 105.

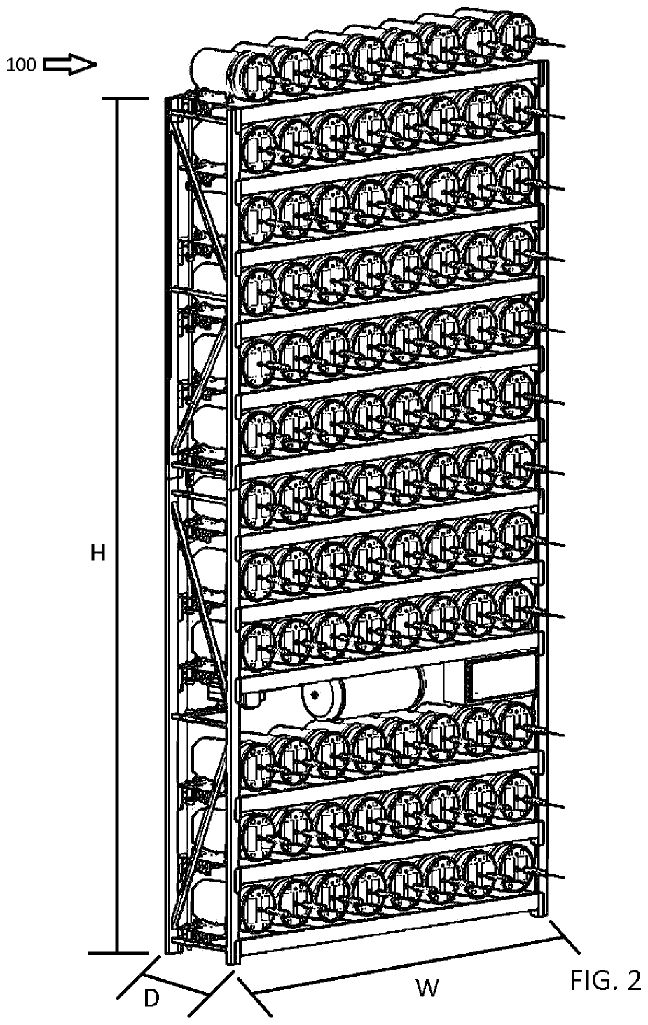

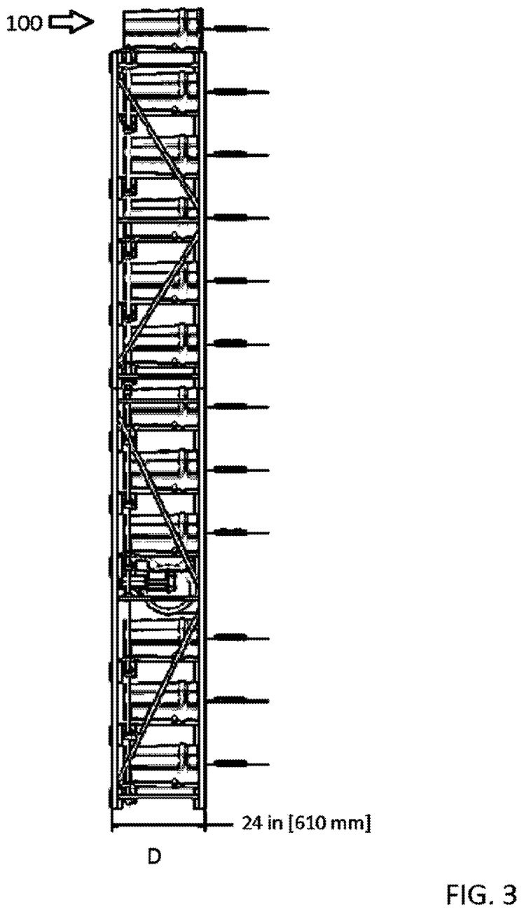

In particular, FIG. 1 provides a front plan view of the racking system 100, FIG. 2 provides a top perspective view of the racking system 100, and FIG. 3 provides a left side elevational view of the racking system 100. The racking system 100 can include a rack 130 configured to dispose a number of containers in various arrangements of horizontal rows and/or vertical columns. The embodiment of the racking system 100 shown includes a rack 130 configured to include twelve rows and eight columns of containers 105. The rack 130 shown for the example racking system 100 depicted can hold up to ninety-six (96) containers. For this rack 130 configuration, a height H of the racking system 100 can be about 216 inches (5486 mm), a width W can be about 108 inches (2743 mm), and a depth D can be about 24 inches (610 mm), to accommodate the particular containers 105 shown. However, it should be appreciated that a skilled artisan can select different configurations and dimensions of the rack 130 for the racking system 100, within the scope of this disclosure, based upon considerations including the configuration of the containers 105, including the size, shape, and number of containers 105, as well as the available space, structure, and geometry of the facility housing the racking system 100.

The racking system 100 can be used with at least one container. As mentioned, in the example shown, the racking system 100 can hold up to ninety-six (96) containers. Different racks for the racking system 100 have different heights H, widths W, and depths D that can change the amount and configuration of the containers 105 configured for the racking system 100. As shown, each of the containers 105 can be stored in a horizontal orientation, where generally cylindrical axis is positioned in a horizontal orientation; e.g., a bucket laying on its side. Each of the containers 105 can be individually connected to an inert gas source 135 via the fluid inlet 115, where the fluid inlet 115 can have a quick connect device that allows for rotation of the container 105 while still being connected to the inert gas source 135. Depicted in the lower portion of the racking system 100, space in one of the rows of the rack 130 can house a drive motor 140 for powering the agitators 125, a surge tank for the inert gas source 135, and a controller 145 that can be interconnected to other computer devices, networks, as well as other racking systems, as provided by the present technology.

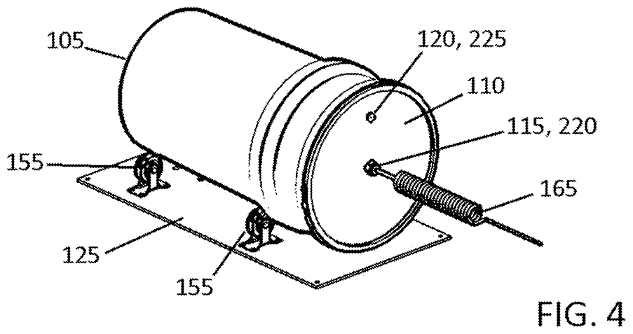

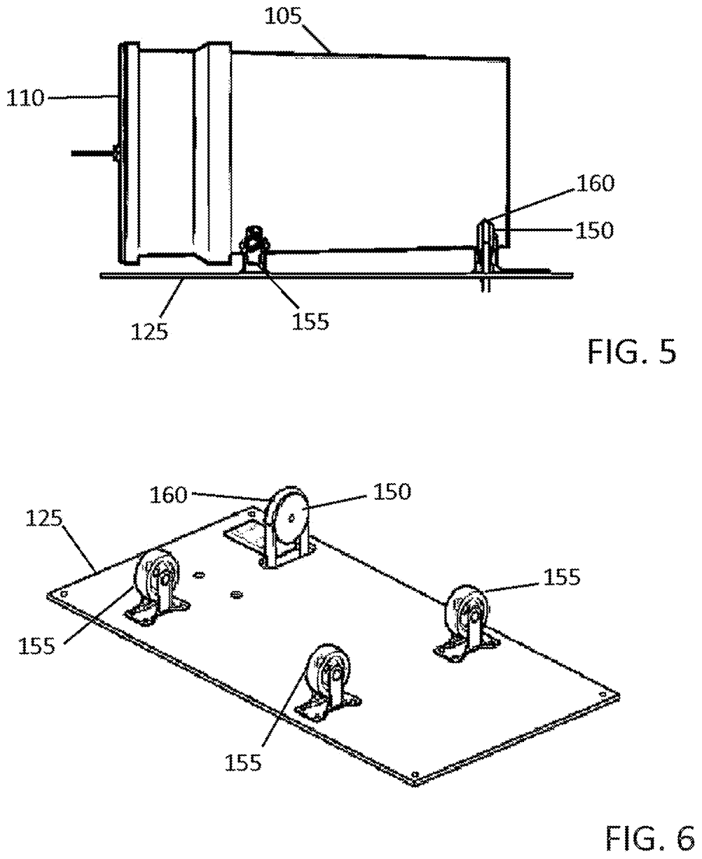

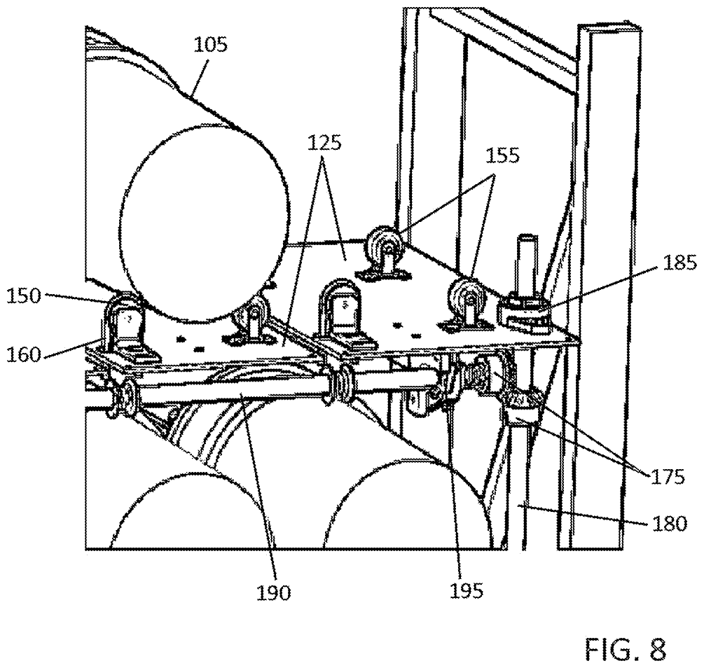

With reference to FIGS. 4-6, the container 105 and the agitator 125 can be cooperatively configured to interact, allowing the agitator 125 to impart the motion unto the container 105. The container 105 in the embodiment shown is configured as plastic cylinder having a removable lid 110, where the cylinder walls can be tapered toward an end opposite to the lid. The particular configuration shown allows the agitator 125 to impart a rotary motion unto the container 105. To this end, the agitator 125 includes a drive wheel 150 configured to contact the container 105 and impart the rotary motion unto the container 105. The drive wheel 150 can be configured to receive a portion of a weight of the container 105. In this way, the container 105 can be partially resting on the drive wheel 150. The agitator 125 can further include three (3) free rolling wheels 155 that cooperate with the drive wheel 150 to fully support the container 105 and maintain the container 105 in place during rotatory motion imparted by the drive wheel 150. The drive wheel 150 can further include a band 160 that can be connected to a horizontal drive shaft 190 (see FIG. 8). The container 105 can therefore rest by gravity upon the three (3) free rolling wheels 155 and the drive wheel 150 of the agitator, where the drive wheel 150 and band 160 can impart motion unto the container 105, which in this case includes rotary motion that rotates the container 105. A right side elevational view of the container 105 is shown in FIG. 5, where one or both of the drive wheel 150 and band 160 can contact the container 105.

The drive wheel 150 and the band 160 can be part of an O-ring line shaft driving system, where the drive wheel 150 remains in contact with the container 105 by gravity. This can allow the container 105 to be rotated in either direction, which can include at least a full rotation of the container. Certain embodiments can include up to 540 degrees of rotation. For example, by rotating up to 540 degrees, the system ensures that the plant product at a bottom of the container 105 is not being crushed by gravity acting on the plant product on top of it within the container 105. In this way, the plant product is not subjected to an environmental gradients or different microclimates within the container 105, where the rotary motion tumbles or mixes the plant product so that an entirety of the plant product more uniformly experiences the controlled environment provided within the container 105, thereby promoting uniform curing of the plant product. The fluid inlet 115 for introducing the fluid into the container 105 can be fluidly coupled to the inert gas source 135 using self-retracting tubing 165 or pigtail tubing that can minimize effects of any twists or kinks imparted thereto based upon the degrees of rotation imparted to the container 105. The fluid inlet 115 can also include an infinitely rotatable coupling. In this way, the container 105 can be rotated as many times as desired in either direction without imparting any twists or kinks to tubing used to fluidly couple the fluid inlet 115 and the inert gas source 135.

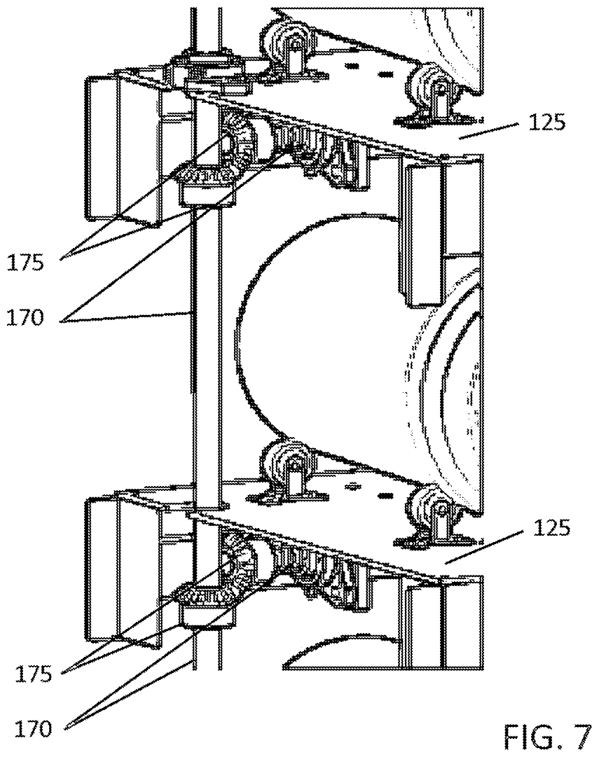

With reference to FIG. 7, depicted is an enlarged partial perspective view of drive shafts 170 coupled via miter gear drive connections 175 to translate power from the drive motor 140 power source to the agitator 125. As shown in FIG. 8, a vertical drive shaft 180 is mounted through vertical shaft bearings 185 and can hold miter gear drive connections 175 in place for each storage row in the racking system 100. The miter gear drive connections 175 transfer motion from the vertical drive shaft 180 to one or more horizontal drive shafts 190 held by horizontal shaft bearings 195. The bands 160 can transfer motion from the horizontal drive shafts 190 to the drive wheels 150 of the agitators 125 of the containers 105.

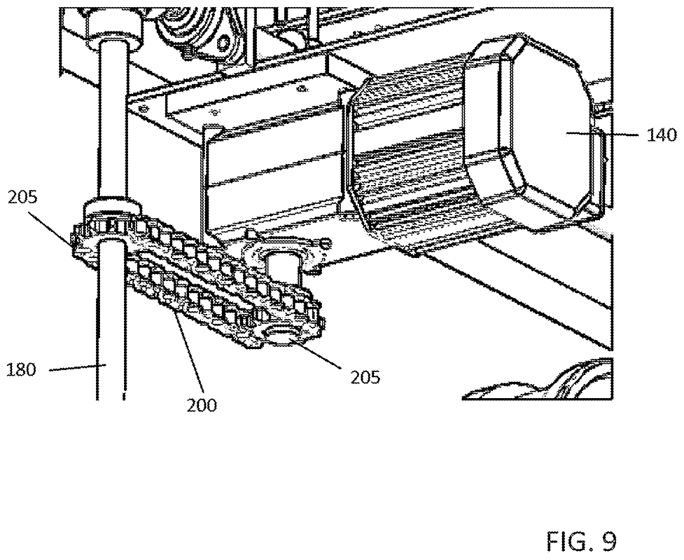

With reference to FIG. 9, depicted is an enlarged partial perspective view of a drive motor 140 coupled to a vertical drive shaft 180 via a chain 200. The drive motor 140 can include an electric motor translating power to the vertical drive shaft 180 using the chain 200 and sprockets 205 associated with the drive shaft 210 and vertical drive shaft 180, respectively. In this way, the rotation of the vertical drive shaft 180 can subsequently rotate all the containers 105 in the racking system 100. The drive motor 140 specifications can be selected based on the size of the racking system 100 to be installed, or where the drive motor 140 is used to multiple racking systems 100. One example includes where the drive motor 140 is a ¼ horsepower 230 Volt AC electric motor capable of 14 rotations per minute.

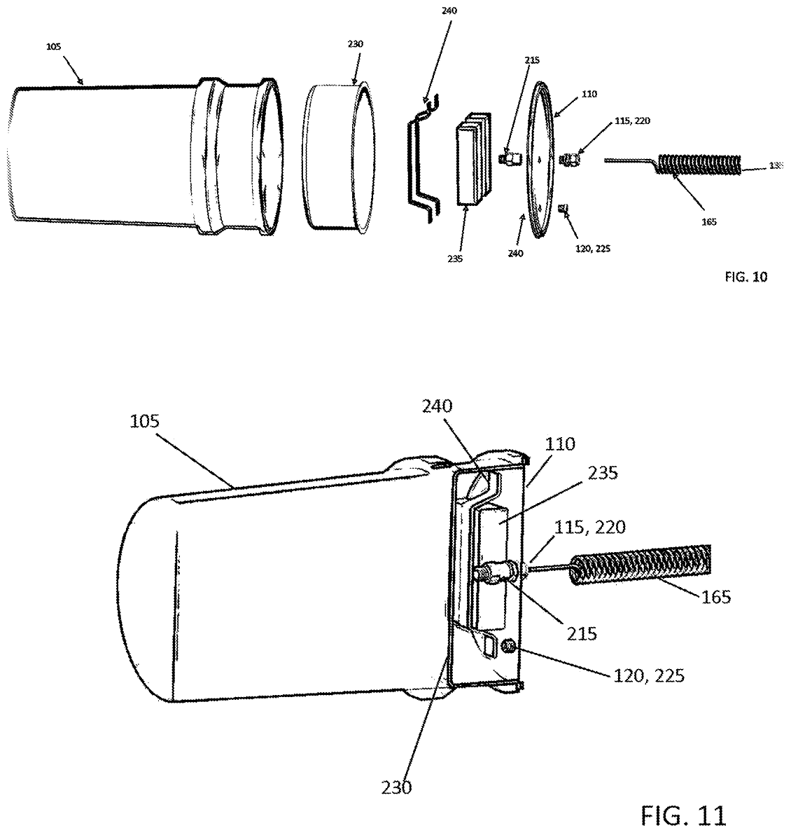

With reference to FIGS. 10-11, depicted are exploded and cross-sectional views a container 105 and related components of the racking system 100, including the lid 110, the fluid inlet 115, the valve 120, and the agitator 125. In the embodiment shown, the container 105 is substantially cylindrical and includes a removable lid 110. The fluid inlet 115 is disposed in the lid 110 and includes a check valve 215. As described herein, the fluid inlet 115 can include an infinitely rotatable coupling 220. The inert gas source 135 can be fluidly coupled to the fluid inlet 115 using self-retracting tubing 165. The valve 120 includes a pressure release valve 225 and the valve 120 is disposed in the lid 110. A screen 230 is provided that is configured to be disposed within the container 105 and prevent the plant product from contacting the lid 110 when the lid 110 fluidly seals the container 105. A humidifying element 235 is provided that is configured to be disposed within the container 105 when the lid 110 fluidly seals the container 105. As shown, the humidifying element 235 can be coupled to an interior face 240 of the lid 110 using fasteners 240. The embodiment shown uses containers 105 configured as 5-gallon or 7-gallon food grade buckets made from food grade high density polyethylene plastic that can withstand temperatures up to 180 degrees F. (82 degrees C.) or food grade metal (e.g., stainless steel or aluminum).

The screen 230 can be configured as a heavy-duty nylon mesh screen that can hold the plant product away from the lid 110 of the container 105 when the assembled container 105 is placed in the horizontal orientation. The screen 230 can be installed before securing the lid 110 to the container 105. The lid 110 can be made of metal and can be designed for easy cleaning and sanitizing when changing storage containers 105. The lid 110 can use various types of the fastener 240 or holder that can be welded to the lid 110 to store one or more humidifying elements 235 configured as hydration packs able to maintain the relative humidity in the container 105 at 58% to 62%. The humidifying elements 235 can be disposed on the interior face 240 of the lid 110 flanking the centrally disposed inline check valve 215 with the fluid inlet 115. The fluid inlet 115 can include nylon, aluminum, brass, or metal adapter fittings to connect the self-retracting tubing 165 (e.g., plastic 6 mm tubing) to the inert gas source 135 to ensure that there is no oxygen that can get into the container 105. Radially located with respect to the fluid inlet 115, toward the outside of the lid 110, the valve 120 including a pressure relief valve 245 is disposed in the lid 110. The valve 120 can be made of nylon, aluminum, brass, or metal can include a pressure relief valve 245 that can be preset to release pressures ranging from 0.25 PSI up to 1 PSI, for example.

The controller 145 can be configured to control the agitator 125 and the motion imparted unto the container 105. The racking system 100 can also include one or more motion sensors (not shown) that can monitor various portions of the racking system 100 related to the motion imparted to the container 105 by the agitator 125. Such motion sensors can be in communication with the controller 145. For example, a motion sensor can be configured to monitor the rotation of the vertical drive shaft 180 at scheduled times, where the rotation motion sensor and the rotation software may be capable of sending notifications of any errors or faults in the racking system 100. The controller 145, and any associated motion sensors, can be interfaced using a computer or can embody a computer, and therefore can allow a user to use automatic and/or manual operating modes of the racking system 100. The controller 145 can also be in communication with various remote devices (e.g., mobile phones, tablets, laptops, etc.), through wired and/or wireless communication, where the controller 145 and/or associated computer(s) can report the operational status of the racking system 100 as well as receive automated instructions, manually inputted instructions, or instruction modifications.

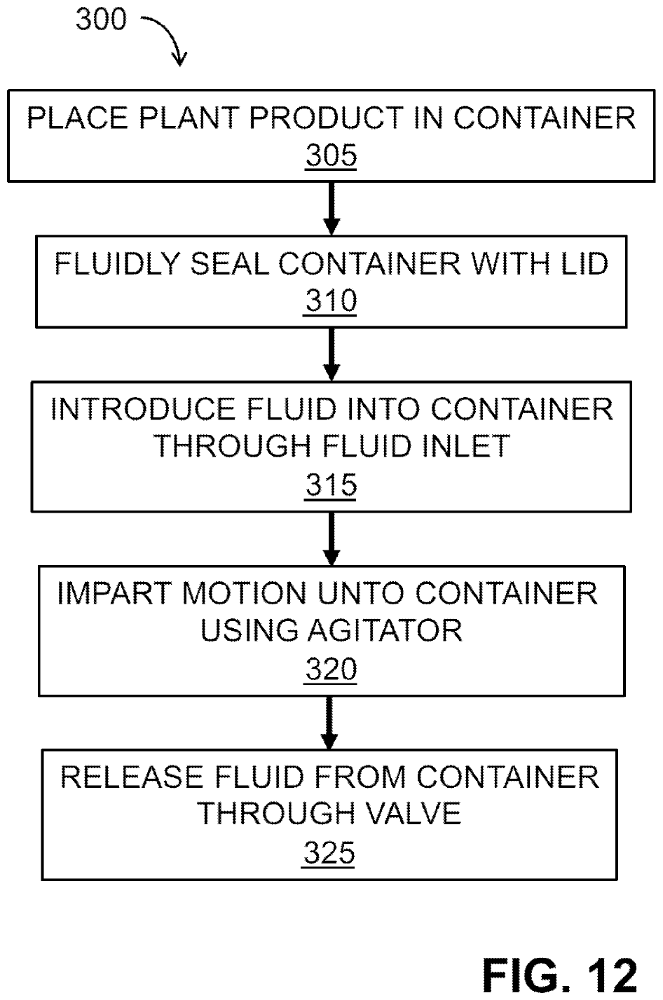

An embodiment of a method of operating a storage and curing racking system is shown in FIG. 12 at 300. One or more racking systems, such as racking system 100 shown in FIGS. 1-11, can be provided. A plant product is then placed into a container which is fluidly sealed with a lid. The fluid (e.g., inert gas) is then introduced into the container through a fluid inlet. Motion is imparted unto the container using an agitator. And fluid is released from the container through a valve.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. A racking system for a plant product, comprising:
a container including a lid configured to fluidly seal the container;
a fluid inlet configured to introduce a fluid into the container;
a valve configured to release the fluid from the container; and
an agitator configured to impart a motion unto the container.

2. The racking system of claim 1, wherein the container is substantially cylindrical.

3. The racking system of claim 1, wherein the fluid inlet is disposed in the lid.

4. The racking system of claim 1, wherein the fluid inlet includes a check valve.

5. The racking system of claim 1, wherein the fluid inlet includes an infinitely rotatable coupling.

6. The racking system of claim 1, further comprising an inert gas source fluidly coupled to the fluid inlet.

7. The racking system of claim 6, wherein the inert gas source is fluidly coupled to the fluid inlet using self-retracting tubing.

8. The racking system of claim 1, wherein the valve includes a pressure release valve.

9. The racking system of claim 1, wherein the valve is disposed in the lid.

10. The racking system of claim 1, wherein the agitator is configured to impart a rotary motion unto the container.

11. The racking system of claim 10, wherein the agitator includes a drive wheel configured to contact the container and impart the rotary motion unto the container.

12. The racking system of claim 11, wherein the drive wheel is configured to receive a portion of a weight of the container.

13. The racking system of claim 1, further comprising a screen configured to be disposed within the container and prevent the plant product from contacting the lid when the lid fluidly seals the container.

14. The racking system of claim 1, further comprising a humidifying element configured to be disposed within the container when the lid fluidly seals the container.

15. The racking system of claim 14, wherein the humidifying element is coupled to an interior face of the lid.

16. The racking system of claim 1, further comprising a controller configured to control the agitator and the motion imparted unto the container.

17. The racking system of claim 1, wherein:
the container is one of a plurality of containers, each container including a lid configured to fluidly seal the container;
the fluid inlet is one of a plurality of fluid inlets, each fluid inlet configured to introduce a fluid into a respective container;
the valve is one of a plurality of valves, each valve configured to release the fluid from a respective container; and
the agitator is one of a plurality of agitators, each agitator configured to impart a motion unto a respective container.

18. The racking system of claim 17, further comprising a rack configured to dispose the plurality of containers in an arrangement selected from a group consisting of: at least one horizontal row, at least one vertical column, and combinations thereof.

19. A racking system for a plant product, comprising:
a plurality of containers, each container including a lid configured to fluidly seal the container;
a fluid inlet disposed in each lid, the fluid inlet configured to introduce a fluid into the container, the fluid inlet including a check valve;
an inert gas source configured to be fluidly coupled to each fluid inlet to provide the fluid;
a valve disposed in each lid, the valve configured to release the fluid from the container, the valve including a pressure release valve;
an agitator configured to impart a motion unto each container;
a screen configured to be disposed within each container and prevent the plant product from contacting the lid when the lid fluidly seals the container;
a humidifying element configured to be disposed within each container when the lid fluidly seals the container;
a controller configured to control the agitator and the motion imparted unto each container; and
a rack configured to dispose the plurality of containers in an arrangement selected from a group consisting of: at least one horizontal row, at least one vertical column, and combinations thereof.

20. A method of storing and curing a plant product, comprising:
providing a racking system, the racking system including:
a container including a lid configured to fluidly seal the container;
a fluid inlet configured to introduce a fluid into the container;
a valve configured to release the fluid from the container; and
an agitator configured to impart a motion unto the container;
placing the plant product in the container;
fluidly sealing the container with the lid;
introducing the fluid into the container through the fluid inlet;
imparting the motion unto the container using the agitator; and
releasing the fluid from the container through the valve.
